(19) United States
(12) Patent Application Publication
(10) Pub. No.: US 2024/0057875 A1
(43) Pub. Date: Feb. 22, 2024

(54) BIOLOGICAL INFORMATION MEASUREMENT DEVICE

(71) Applicant: OMRON HEALTHCARE Co., Ltd., Kyoto (JP)

(72) Inventors: Kentaro MORI, Kyoto (JP); Takeshi KUBO, Kyoto (JP); Takashi ONO, Kyoto (JP); Masaki HARADA, Kyoto (JP)

(21) Appl. No.: 18/487,638

(22) Filed: Oct. 16, 2023

Related U.S. Application Data
(63) Continuation of application No. PCT/JP2021/020459, filed on May 28, 2021.

(51) Int. Cl. A61B 5/0205; A61B 5/00; A61B 5/022; A61B 5/28; A61B 5/263

(52) U.S. Cl. CPC A61B 5/0205; A61B 5/681; A61B 5/02233; A61B 5/28; A61B 5/7203; A61B 5/263; A61B 2562/164; A61B 2560/0468

(57) ABSTRACT

A biological information measurement device used by being attached to a wrist of a human body, including a main body including at least a power source unit, an electrocardiographic waveform measurement unit that measures an electrocardiographic waveform of the human body based on a first electrode, a second electrode, and a potential difference when the first electrode and the second electrode are in contact with the human body, a first cuff that applies a force compressing an artery present in the wrist in a state of being attached to the wrist, a second cuff located on a side closer to the wrist than the first cuff in the state of being attached to the wrist and detecting a pressure change caused by pulsation of the artery, a pressing unit disposed between the first cuff and the second cuff, and a blood pressure measurement unit.

### CROSS-REFERENCE TO RELATED APPLICATIONS

This application is the U.S. national stage application filed pursuant to 35 U.S.C. 365(c) and 120 as a continuation of International Patent Application No. PCT/JP2021/020459, filed May 28, 2021, which application is incorporated herein by reference in its entirety.

### TECHNICAL FIELD

The present invention belongs to the technical field related to healthcare, and particularly relates to a biological information measurement device.

### BACKGROUND ART

In recent years, it has become common for information (hereinafter, also referred to as biological information) related to one's body and health, such as a blood pressure value and an electrocardiographic waveform, to be measured by oneself on a daily basis using a measurement device to utilize the measurement result for health management. For this reason, there has been an increasing demand for devices that place importance on portability, with many portable measurement devices being proposed, such as portable devices that can measure both a blood pressure value and an electrocardiographic waveform (for example, Patent Document 1).

Patent Document 1 describes a portable cardiovascular measurement device including a cuff for blood pressure measurement and an electrode for electrocardiographic measurement. According to the invention, a user can measure the blood pressure value and the electrocardiographic waveform at any time by carrying the device.

### CITATION LIST

### Patent Literature

Patent Document 1: JP 2014-36843 A

### SUMMARY OF INVENTION

### Technical Problem

However, in the technique described in Patent Document 1, when the cuff is inflated to measure blood pressure, it is difficult to stably bring the electrode into contact with a surface of the human body, and thus there is a problem that the blood pressure and the electrocardiographic waveform cannot be simultaneously and accurately measured.

In view of the above-described problems, it is an object of the present invention to provide a portable biological information measurement device capable of simultaneously and accurately measuring a blood pressure value and an electrocardiographic waveform.

### Solution to Problem

In order to solve the above-described problems, a biological information measurement device according to the present invention is a biological information measurement device used by being attached to a wrist of a human body, the biological information measurement device including a main body including at least a power source unit, a first electrode, a second electrode, an electrocardiographic waveform measurement unit that measures an electrocardiographic waveform of the human body based on a potential difference when the first electrode and the second electrode are in contact with the human body, a first cuff that applies a force compressing an artery present in the wrist in a state of being attached to the wrist, a second cuff located on a side closer to the wrist than the first cuff in the state of being attached to the wrist and detecting a pressure change caused by pulsation of the artery, a pressing unit disposed between the first cuff and the second cuff and pressing the second cuff when the force compressing the artery is applied by the first cuff in the state of being attached to the wrist, and a blood pressure measurement unit that measures a blood pressure of the human body based on an internal pressure of the second cuff. The second electrode is provided on the pressing unit so as to be capable of coming into contact with the wrist when the force compressing the artery is applied by the first cuff in the state of being attached to the wrist.

It should be noted that the “pressing unit” may be an independent member or may be provided integrally with any other configuration. According to the above-described configuration, since the second electrode is provided in the pressing unit that presses the second cuff, the second electrode can be stably brought into contact with a surface of the human body even at a time of blood pressure measurement, unlike a case where the second electrode is provided in a cuff or a main body case. As a result, the blood pressure can be accurately measured by two types of cuffs, the first cuff and the second cuff, and the electrocardiographic waveform can be simultaneously and accurately measured.

The biological information measurement device may further include a third electrode that removes noise from the electrocardiographic waveform. With such a configuration, noise at the time of electrocardiographic measurement can be reduced, and the electrocardiographic waveform can be measured with higher accuracy.

In addition, the third electrode may be provided on the pressing unit so as to be capable of coming into contact with the wrist when the force compressing the artery is applied by the first cuff in the state of being attached to the wrist.

Further, the pressing unit may be made of a conductive material and may serve as the second electrode. With such a configuration, the number of parts of the device can be reduced.

In addition, the pressing unit may be formed on a surface of the first cuff facing the second cuff. By forming a part of the first cuff as the pressing unit instead of forming the pressing unit as an independent member, the number of parts of the device can be reduced.

The biological information measurement device may have a configuration in which the second electrode is disposed on an inside of the first cuff and the main body is disposed on an outside of the first cuff with a side close to the wrist as the inside. The first cuff may include a wiring connecting portion that routes wiring electrically connecting at least the second electrode and the main body to each other between the inside and the outside of the first cuff.

Although the second electrode needs to be disposed on a side (hereinafter also referred to as the inside) coming into contact with the human body, the main body is disposed on a side (hereinafter also referred to as the outside) opposite to the inside. For this reason, it is necessary to route a conductive wire electrically connecting the electrode and (the control unit of) the main body to each other on the inside and the outside of the device. However, with the configuration of the first cuff as described above, such wiring can be easily performed.

Further, the wiring may be provided on a flexible substrate. Further, the flexible substrate may serve as the pressing unit. With such a configuration, the number of parts of the device can be reduced.

Also, the configurations described above can be combined with one another to constitute the present invention unless the combination leads to contradiction.

### Advantageous Effects of Invention

According to the present invention, the present invention can provide a portable biological information measurement device capable of simultaneously and accurately measuring a blood pressure value and an electrocardiographic waveform.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a schematic view illustrating a biological information measurement device according to Example 1;

FIG. 2 is an explanatory view illustrating an arrangement relationship between components when the biological information measurement device according to Example 1 is attached;

FIG. 3 is a functional block diagram illustrating a functional configuration of the biological information measurement device according to Example 1;

FIG. 4(A) is a first explanatory view illustrating a structure of a cuff assembly portion of the biological information measurement device according to Example 1;

FIG. 4(B) is a second explanatory view illustrating the structure of the cuff assembly portion of the biological information measurement device according to Example 1;

FIG. 4(C) is a third explanatory view illustrating the structure of the cuff assembly portion of the biological information measurement device according to Example 1;

FIG. 5 is a plan view illustrating a structure of a compression cuff of the biological information measurement device according to Example 1;

FIG. 6(A) is a first explanatory view illustrating the structure of the cuff assembly portion of the biological information measurement device according to a modified example of Example 1;

FIG. 6(B) is a second explanatory view illustrating the structure of the cuff assembly portion of the biological information measurement device according to the modified example of Example 1;

FIG. 6(C) is a third explanatory view illustrating the structure of the cuff assembly portion of the biological information measurement device according to the modified example of Example 1;

FIG. 7(A) is a first explanatory view illustrating the structure of the cuff assembly portion of the biological information measurement device according to another modified example of Example 1;

FIG. 7(B) is a second explanatory view illustrating the structure of the cuff assembly portion of the biological information measurement device according to another modified example of Example 1;

FIG. 7(C) is a third explanatory view illustrating the structure of the cuff assembly portion of the biological information measurement device according to another modified example of Example 1;

FIG. 8(A) is a first explanatory view illustrating the structure of the cuff assembly portion of the biological information measurement device according to still another modified example of Example 1;

FIG. 8(B) is a second explanatory view illustrating the structure of the cuff assembly portion of the biological information measurement device according to still another modified example of Example 1;

FIG. 8(C) is a third explanatory view illustrating the structure of the cuff assembly portion of the biological information measurement device according to still another modified example of Example 1;

FIG. 9 is a functional block diagram illustrating a functional configuration of a biological information measurement device according to Example 2;

FIG. 10(A) is a first explanatory view illustrating the structure of the cuff assembly portion of the biological information measurement device according to Example 2;

FIG. 10(B) is a second explanatory view illustrating the structure of the cuff assembly portion of the biological information measurement device according to Example 2; and,

FIG. 10(C) is a third explanatory view illustrating the structure of the cuff assembly portion of the biological information measurement device according to Example 2.

### DESCRIPTION OF EMBODIMENTS

### Example 1

Embodiments of the present invention will be specifically described below with reference to the drawings. It should be noted that the dimension, material, shape, relative arrangement and the like of the components described in the present embodiment are not intended to limit the scope of this invention to them alone, unless otherwise stated.

Overall Configuration of Device

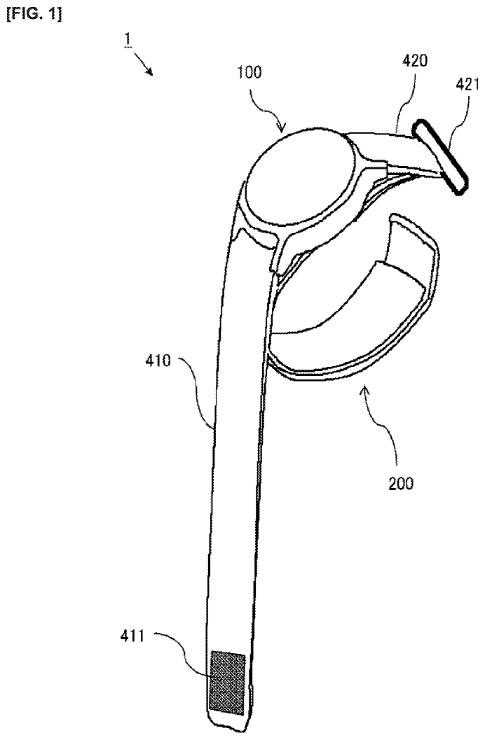

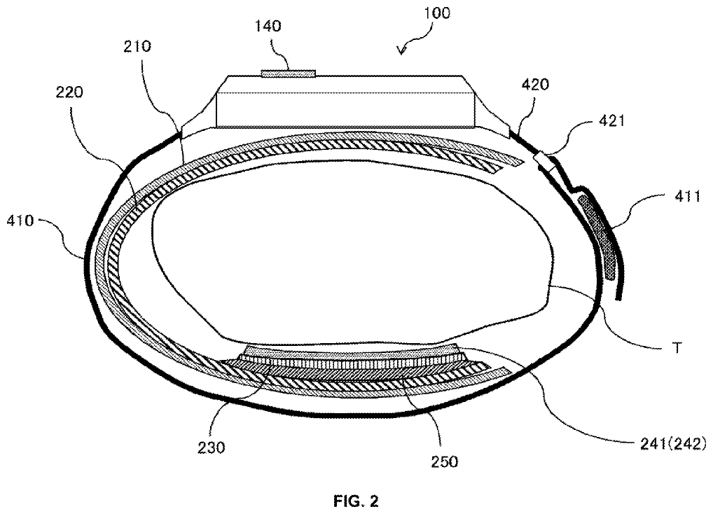

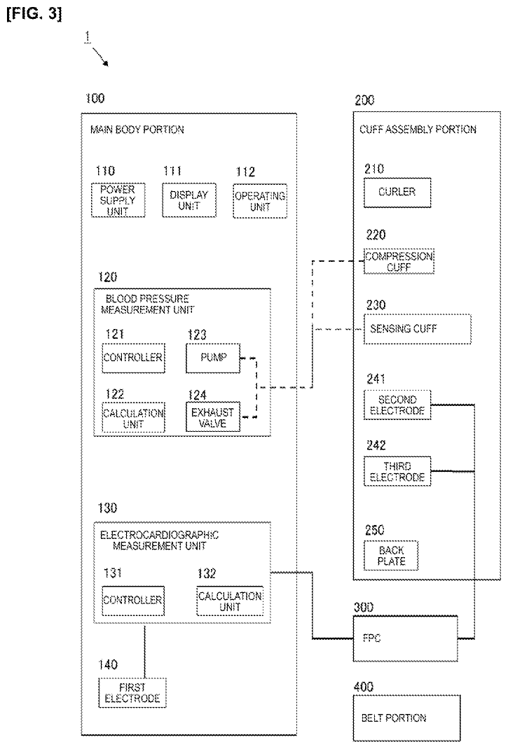

FIG. 1 is a schematic view illustrating an appearance configuration of a biological information measurement device 1 according to the present example. FIG. 2 is an explanatory view illustrating an arrangement relationship between components when the biological information measurement device 1 according to the present example is attached to a wrist. FIG. 3 is a functional block diagram illustrating a functional configuration of the biological information measurement device 1 according to the present example.

As illustrated in FIG. 1 to FIG. 3, the biological information measurement device 1 generally includes a main body portion 100, a cuff assembly portion 200, and a belt portion 400, and can measure a blood pressure value and an electrocardiographic waveform in a state of being attached to a wrist of a human body. The belt portion 400 includes a first belt portion 410 including a hook-and-loop fastener 411 including a hook, and a second belt portion 420 including a belt passing ring 421. When the biological information measurement device 1 is attached, the first belt portion 410 is wound around the wrist and inserted through the belt passing ring 421, and the hook-and-loop fastener 411 is attached to any position of the first belt portion 410 (where a loop with which a hook engages is formed) to fix the biological information measurement device 1. FIG. 2 illustrates an arrangement relationship between components in a state in which the biological information measurement device 1 is attached to a wrist T in this manner. The biological information measurement device 1 includes a flexible printed circuit (FPC) 300 (not illustrated in FIG. 1 and FIG. 2) on which wiring for electrically connecting an electrocardiographic measurement unit 130 of the main body portion 100 and a second electrode 241 and a third electrode 242 of the cuff assembly portion 200 to each other is disposed.

As illustrated in FIG. 3, the main body portion 100 includes a housing, a power source unit 110, a display unit 111, an operation unit 112, a blood pressure measurement unit 120, the electrocardiographic measurement unit 130, and a first electrode 140.

The power source unit 110 includes a battery that supplies the power required for operation of the device. The battery may be, for example, a secondary battery such as a lithium ion battery, or a primary battery.

The display unit 111 includes a display device such as a liquid crystal display, and may be provided with an LED indicator or the like. Although the operation unit 112 includes, for example, various operation buttons such as a power button, the display unit 111 and the operation unit 112 may be integrated such as in a touch panel display.

The blood pressure measurement unit 120 is a functional unit that controls the cuff assembly portion 200 described later, and measures the blood pressure of the user based on information obtained by the cuff assembly portion 200, and includes a control unit 121, a calculation unit 122, a pump 123, and an exhaust valve 124. The control unit 121 and the calculation unit 122 include, for example, a central processing unit (CPU) or the like, and although not illustrated, may include a storage unit including a random access memory (RAM) or the like.

The control unit 121 is a functional unit that controls the blood pressure measurement unit 120, controls a cuff pressure of the cuff assembly portion 200 via the calculation unit 122, the pump 123, and the like, and obtains information for measuring the blood pressure of the user from the artery in the wrist to which the biological information measurement device 1 is attached. The calculation unit 122 measures the blood pressure value based on the information obtained in this manner. The pump 123 and the exhaust valve 124 are mechanisms for supplying and exhausting air to and from a compression cuff 220 and a sensing cuff 230 described later.

The electrocardiographic measurement unit 130 is a functional unit that measures an electrocardiographic waveform of the user based on a potential difference between the first electrode 140 and the second electrode 241 in contact with a surface of the human body, and includes a control unit 131 and a calculation unit 132. The control unit 131 and the calculation unit 132 include the above-described CPU or the like. From the viewpoint of hardware, the control unit 131 and the calculation unit 132 may have the same configuration as the control unit 121 and the calculation unit 122 of the blood pressure measurement unit 120.

Each of the blood pressure measurement unit 120 and the electrocardiographic measurement unit 130 includes an AD conversion circuit, an amplifier, a filter, and the like (not illustrated) in addition to the CPU, the RAM, and the like described above. However, these are configured by a known technique, and thus description thereof is omitted.

The cuff assembly portion 200 includes a curler 210, the compression cuff 220, the sensing cuff 230, the second electrode 241, the third electrode 242, and a back plate 250. The curler 210 is a member serving as a base for holding the compression cuff 220, and as illustrated in FIG. 2, is disposed on the outermost side (that is, the side away from the wrist) of the cuff assembly portion 200.

The compression cuff 220 is inflated by the air sent from the pump 123 to tighten the wrist T of an attachment portion and has a role of applying external pressure to the artery (not illustrated) present in the wrist T. The sensing cuff 230 is a fluid bag for detecting a pressure applied to a portion compressed by the compression cuff 220, and the pressure applied to the compressed portion is measured by detecting an internal pressure of the sensing cuff 230 with a pressure meter (not illustrated) in a state where a small amount of air is contained in the sensing cuff 230. The back plate 250 is a flexible flat plate-like member disposed between the compression cuff 220 and the sensing cuff 230, suppresses excessive bending of the sensing cuff 230 at the time of compression by the compression cuff 220, and equalizes a pressure distribution in the sensing cuff 230. That is, in the present example, the back plate 250 corresponds to the pressing unit.

Each of the second electrode 241 and the third electrode 242 is an electrode disposed at a position capable of coming into contact with a surface of the human body, the second electrode 241 is an electrode for electrocardiographic waveform measurement, and the third electrode 242 functions as a neutral (right foot) electrode for outputting a feedback signal for noise removal.

Structure of Cuff Assembly Portion

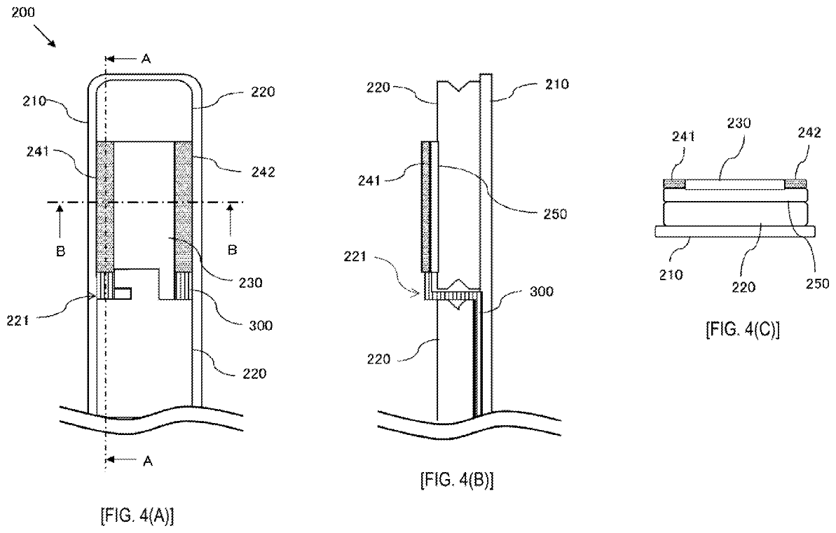

Next, a structure of the cuff assembly portion 200 will be described with reference to FIG. 4(A), FIG. 4(B) and FIG. 4(C). FIG. 4(A) is a schematic plan view when the cuff assembly portion 200 according to the present example is viewed from the inside, FIG. 4(B) is a schematic view illustrating an A-A cross section in FIG. 4(A), and FIG. 4(C) is a schematic view illustrating a B-B cross section in FIG. 4(A). As illustrated in FIG. 4(A), FIG. 4(B) and FIG. 4(C), the cuff assembly portion 200 has a configuration in which the compression cuff 220, the back plate 250, and the sensing cuff 230 are stacked in this order with the curler 210 as the outermost side. The second electrode 241 and the third electrode 242 are provided on the back plate 250, and are disposed at both ends of the sensing cuff 230 in the lateral direction.

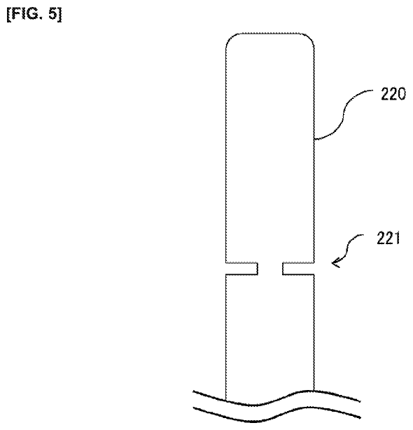

The second electrode 241 and the third electrode 242 are connected to a FPC 300 provided with the conductive wire, and the FPC 300 is connected to the control unit 131 of the main body portion 100 (not illustrated), and thus functions as wiring electrically connecting the control unit 131 and the electrodes to each other. Here, the second electrode 241 and the third electrode 242 are disposed on the innermost side (that is, the side closest to the wrist) of the cuff assembly portion 200, whereas the main body portion 100 is disposed on the side farthest from the wrist, and thus the compression cuff 220 is provided with a wiring connecting portion 221 for routing the FPC 300 from the inside to the outside. FIG. 5 illustrates a configuration of the wiring connecting portion 221 in the compression cuff 220. As illustrated in FIG. 5, the wiring connecting portion 221 has a structure cut out from both sides so as to leave the vicinity of the center portion of the compression cuff 220 in the lateral direction. As illustrated in FIG. 4(A), FIG. 4(B) and FIG. 4(C), the FPC 300 is inserted into the cutouts, and thus routed between the inside and the outside of the cuff assembly portion 200.

Measurement of Biological Information

In order to measure biological information by the biological information measurement device 1 having the above-described configuration, first, the cuff assembly portion 200 and the first belt portion 410 are wound around the wrist such that the main body portion 100 faces the back side of the hand. Then, the first belt portion 410 is passed through the belt passing ring 421 of the second belt portion 420 and folded back, and the hook-and-loop fastener 411 of the first belt portion 410 is attached to any position of the first belt portion 410 and the biological information measurement device 1 is attached to the wrist. At this time, the biological information measurement device 1 is attached such that the sensing cuff 230, the second electrode 241, and the third electrode 242 are located on the palm side of the wrist.

Then, measurement is started by operating the operation unit 112. Specifically, air is injected into the compression cuff 220 to compress (the artery of) the wrist T, the artery is occluded to temporarily stop the blood flow, and then the air is gradually discharged from the compression cuff 220 to restore the blood flow in the artery, and the pressure at that time is measured by the sensing cuff 230. Blood pressure measurement by a so-called oscillometric method is performed.

When the wrist is compressed by the compression cuff 220 at the time of the blood pressure measurement, the second electrode 241 and the third electrode 242 are in contact with (pressed against) the surface of the wrist. Thus, by touching the first electrode 140 provided in the housing of the main body portion 100 with a finger to which the biological information measurement device 1 is not attached, the electrocardiographic waveform can be measured by a so-called I induction method based on the potential difference between the first electrode 140 and the second electrode 241.

As described above, according to the biological information measurement device 1 of the present example, the blood pressure value and the electrocardiographic waveform can be simultaneously and accurately measured by the portable device of a type to be attached to the wrist.

### Modified Example 1

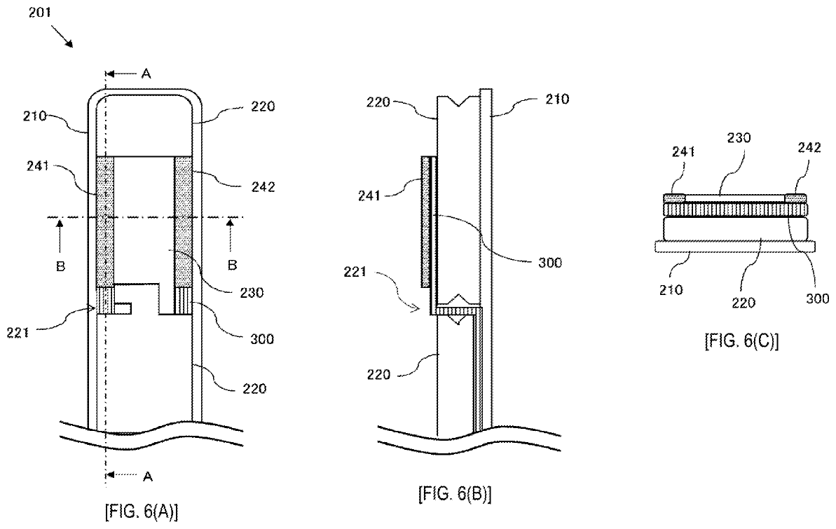

Although the cuff assembly portion 200 of the above-described Example 1 includes the back plate 250, the cuff assembly portion need not necessarily include the back plate. FIG. 6(A), FIG. 6(B) and FIG. 6(C) illustrate a structure of a cuff assembly portion 201 according to a modified example of Example 1. In FIG. 6(A), FIG. 6(B) and FIG. 6(C), the same components as those in the cuff assembly portion 200 in Example 1 are denoted by the same reference numerals, and detailed description thereof will be omitted.

FIG. 6(A) is a schematic plan view when the cuff assembly portion 201 according to the present modified example is viewed from the inside, FIG. 6(B) is a schematic view illustrating an A-A cross section in FIG. 6(A), and FIG. 6(C) is a schematic view illustrating a B-B cross section in FIG. 6(A). As illustrated in FIG. 6(A), FIG. 6(B) and FIG. 6(C), the back plate does not present if the cuff assembly portion 201 according to the present modified example. Instead, the FPC 300 extends immediately outside the sensing cuff 230 and the second electrode 241 and third electrode 242 are also formed on the FPC 300.

That is, in the present modified example, the FPC 300 serves as a role of the back plate and corresponds to the pressing unit. Thus, the back plate as a member can be omitted, contributing to a reduction in the number of parts of the device.

### Modified Example 2

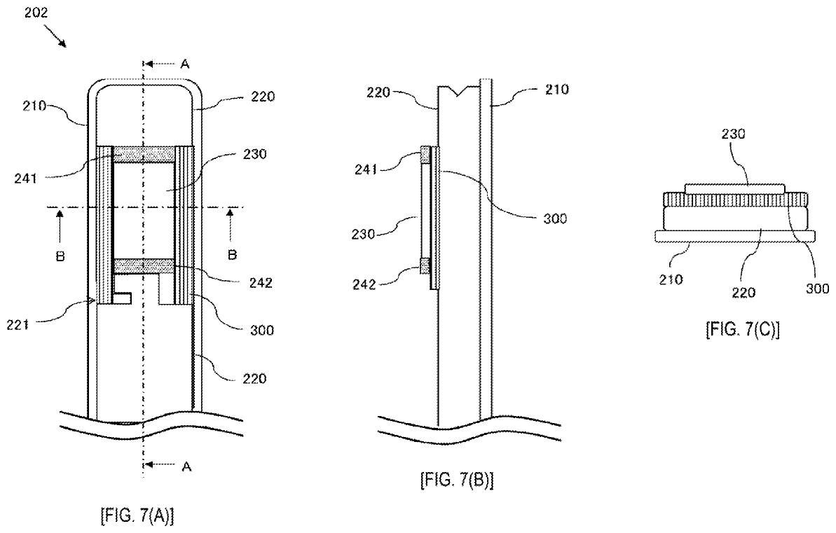

In addition, in the cuff assembly portion, the second electrode 241 and the third electrode 242 need not necessarily be disposed at both ends of the sensing cuff 230 in the lateral direction, and may be disposed at both ends in the longitudinal direction. FIG. 7(A), FIG. 7(B) and FIG. 7(C) illustrate a structure of a cuff assembly portion 202 according to such a modified example.

FIG. 7(A) is a schematic plan view when the cuff assembly portion 202 according to the present modified example is viewed from the inside, FIG. 7(B) is a schematic view illustrating an A-A cross section in FIG. 7(A), and FIG. 7(C) is a schematic view illustrating a B-B cross section in FIG. 7(A). As illustrated in FIG. 7(A), FIG. 7(B) and FIG. 7(C), the cuff assembly portion 202 in the present modified example is provided such that the second electrode 241 and the third electrode 242 are located on the FPC 300 and at both ends of the sensing cuff 230 in the longitudinal direction. That is, also in the present modified example, the FPC 300 is the pressing unit serving as the role of the back plate.

### Modified Example 3

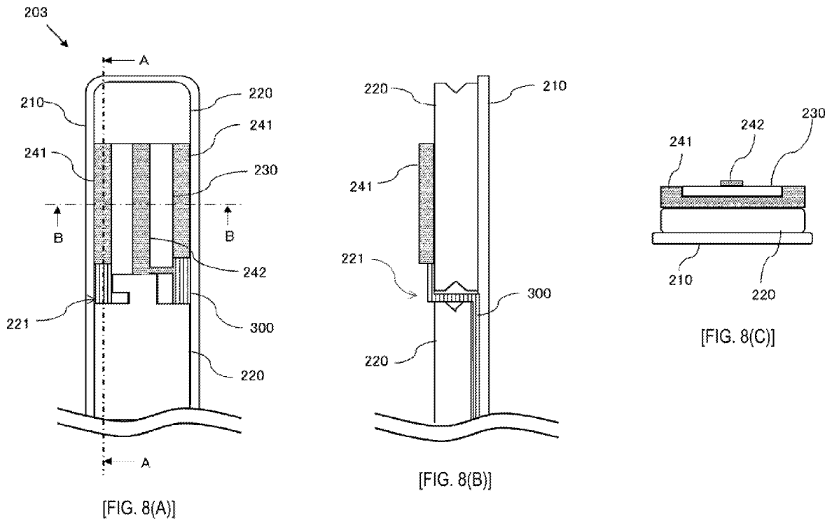

In addition, the second electrode 241 and the third electrode 242 may have arrangements and structures other than the above-described examples. FIG. 8(A), FIG. 8(B) and FIG. 8(C) illustrate a structure of a cuff assembly portion 203 according to a third modified example of Example 1. FIG. 8(A) is a schematic plan view when the cuff assembly portion 203 according to the present modified example is viewed from the inside, FIG. 8(B) is a schematic view illustrating an A-A cross section in FIG. 8(A), and FIG. 8(C) is a schematic view illustrating a B-B cross section in FIG. 8(A).

As illustrated in FIG. 8(A), FIG. 8(B) and FIG. 8(C), the cuff assembly portion 203 in the present modified example is formed in a shape surrounding the sensing cuff 230 from three sides in FIG. 8(C) while the second electrode 241 is in contact with a surface on the outside and both end surfaces of the sensing cuff 230 in the lateral direction. That is, in the present modified example, the second electrode 241 can also be regarded as the back plate made of a conductive material, and corresponds to the pressing unit. On the other hand, the third electrode 242 is formed on a surface on the inside of the sensing cuff 230 so as to longitudinally traverse the central portion of the sensing cuff 230 in the lateral direction. Even with such a structure of the cuff assembly portion 203 according to the present modified example, the number of parts of the device can be reduced.

### Example 2

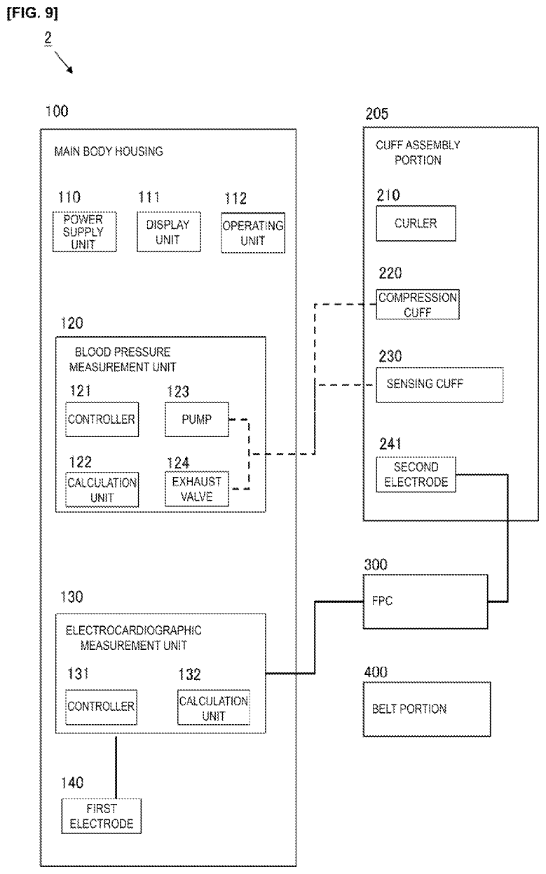

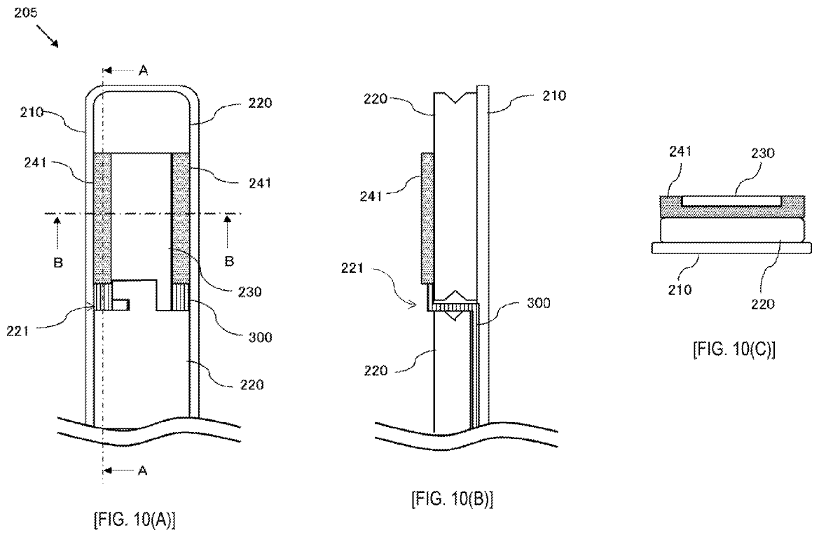

In the above-described Example 1, the cuff assembly portion includes the second electrode and the third electrode. However, the cuff assembly portion may have other configurations. Other examples according to the present invention will be described below with reference to FIG. 9, FIG. 10(A), FIG. 10(B), and FIG. 10(C). A biological information measurement device 2 according to the present example has configurations similar in appearance and the like to those of the biological information measurement device 1 according to Example 1, and such configurations are denoted by the same reference numerals as those of Example 1 and will not be described.

FIG. 9 is a functional block diagram illustrating a functional configuration of the biological information measurement device 2 according to the present example, FIG. 10(A) is a schematic plan view of a cuff assembly portion 205 of the biological information measurement device 2 according to the present example when viewed from the inside, FIG. 10(B) is a schematic view illustrating an A-A cross section in FIG. 10(A), and FIG. 10(C) is a schematic view illustrating a B-B cross section in FIG. 10(A).

As illustrated in FIG. 9, FIG. 10(A), FIG. 10(B), and FIG. 10(C), the third electrode and the back plate are not provided in the cuff assembly portion 205 of the biological information measurement device 2. Similarly to the case of the modified Example 3 of Example 1, the second electrode 241 is formed in a shape surrounding the sensing cuff 230 from three sides in FIG. 10(C) while being in contact with a surface on the outside and both end surfaces of the sensing cuff 230 in the lateral direction. That is, in the present example, the second electrode 241 also serves as the back plate, that is, the pressing unit.

Even with a configuration in which the third electrode is omitted as in the biological information measurement device 2, the blood pressure and the electrocardiographic waveform can be simultaneously measured similarly to the case of Example 1. Such a configuration in which the third electrode is omitted can simplify the device configuration and contribute to a reduction in manufacturing cost.

Other Points

The description of the examples described above is merely illustrative of the present invention, and the present invention is not limited to the specific embodiments described above. Within the scope of the technical idea of the present invention, various modifications and combinations may be made. For example, in the structure of the cuff assembly portion of Example 1 and modified Examples 1 and 2 of Example 1, the third electrode may be omitted.

In addition, the wiring connecting portion 221 of the compression cuff 220 is not limited to the structure in which the cutouts are formed from both ends, but may be a structure in which an opening is partially provided, or the compression cuff 220 may be separated into a first compression cuff portion and a second compression cuff portion, and the separated portion may be used as the wiring connecting portion 221.

In addition, the compression cuff 220 may be disposed only on the back side of the wrist (that is, the side facing the sensing cuff via the wrist) and the wiring connecting portion 221 may not be provided.

Further, means for fixing the device to the wrist is not limited to the hook-and-loop fastener 411. As in a general wristwatch, the first belt portion may be provided with a plurality of holes and the second belt portion may be provided with a buckle and a prong, or any other desired fastening means may be used. In addition, the present invention can be applied to a device which is not necessarily intended to be carried.

### REFERENCE NUMERALS LIST

1,2 Biological information measurement device
100 Main body portion
110 Power source unit
111 Display unit
112 Operation unit
120 Blood pressure measurement unit
121 Control unit
122 Calculation unit
123 Pump
124 Exhaust valve
130 Electrocardiographic measurement unit
131 Control unit
132 Calculation unit
140 First electrode
200, 201, 202, 203, 205 Cuff assembly portion
210 Curler
220 Compression cuff
221 Wiring connecting portion
230 Sensing cuff
241 Second electrode
242 Third electrode
250 Back plate
300 FPC
400 Belt portion
410 First belt portion
411 Hook-and-loop fastener
420 Second belt portion
421 Belt passing ring
T Wrist

What is claimed is:

1. A biological information measurement device used by being attached to a wrist of a human body, the biological information measurement device comprising:
a main body including at least a power source unit;
a first electrode;
a second electrode;
an electrocardiographic waveform measurement unit configured to measure an electrocardiographic waveform of the human body based on a potential difference when the first electrode and the second electrode are in contact with the human body;
a first cuff configured to apply a force compressing an artery present in the wrist in a state of being attached to the wrist;
a second cuff located on a side closer to the wrist than the first cuff in the state of being attached to the wrist and configured to detect a pressure change caused by pulsation of the artery;
a pressing unit disposed between the first cuff and the second cuff and configured to press the second cuff when the force compressing the artery is applied by the first cuff in the state of being attached to the wrist; and
a blood pressure measurement unit configured to measure a blood pressure of the human body based on an internal pressure of the second cuff, wherein
the second electrode is provided on the pressing unit so as to be capable of coming into contact with the wrist when the force compressing the artery is applied by the first cuff in the state of being attached to the wrist.

2. The biological information measurement device according to claim 1, further comprising a third electrode configured to remove noise from the electrocardiographic waveform.

3. The biological information measurement device according to claim 2, wherein the third electrode is provided on the pressing unit so as to be capable of coming into contact with the wrist when the force compressing the artery is applied by the first cuff in the state of being attached to the wrist.

4. The biological information measurement device according to claim 1, wherein the pressing unit is made of a conductive material and serves as the second electrode.

5. The biological information measurement device according to claim 1, wherein the pressing unit is formed on a surface of the first cuff facing the second cuff.

6. The biological information measurement device according to claim 1, wherein
the biological information measurement device has a configuration in which the second electrode is disposed on an inside of the first cuff and the main body is disposed on an outside of the first cuff with a side close to the wrist as the inside, and
the first cuff includes a wiring connecting portion configured to route wiring electrically connecting at least the second electrode and the main body to each other between the inside and the outside of the first cuff.

7. The biological information measurement device according to claim 6, wherein the wiring is provided on a flexible substrate.

8. The biological information measurement device according to claim 7, wherein the flexible substrate serves as the pressing unit.

9. The biological information measurement device according to claim 2, wherein the pressing unit is made of a conductive material and serves as the second electrode.

10. The biological information measurement device according to claim 3, wherein the pressing unit is made of a conductive material and serves as the second electrode.

11. The biological information measurement device according to claim 2, wherein the pressing unit is formed on a surface of the first cuff facing the second cuff.

12. The biological information measurement device according to claim 3, wherein the pressing unit is formed on a surface of the first cuff facing the second cuff.

13. The biological information measurement device according to claim 2, wherein
the biological information measurement device has a configuration in which the second electrode is disposed on an inside of the first cuff and the main body is disposed on an outside of the first cuff with a side close to the wrist as the inside, and
the first cuff includes a wiring connecting portion configured to route wiring electrically connecting at least the second electrode and the main body to each other between the inside and the outside of the first cuff.

14. The biological information measurement device according to claim 3, wherein
the biological information measurement device has a configuration in which the second electrode is disposed on an inside of the first cuff and the main body is disposed on an outside of the first cuff with a side close to the wrist as the inside, and
the first cuff includes a wiring connecting portion configured to route wiring electrically connecting at least the second electrode and the main body to each other between the inside and the outside of the first cuff.
